(19) United States
(12) Patent Application Publication
(10) Pub. No.: US 2020/0129482 A1
(43) Pub. Date: Apr. 30, 2020

(54) TREATMENT OF NON-SMALL CELL LUNG CANCER

(71) Applicant: AbbVie Inc., North Chicago, IL (US)

(72) Inventors: Peter Ansell, Grayslake, IL (US); Lei He, Northbrook, IL (US); Xin Huang, Glenview, IL (US); Vasudha Sehgal, North Chicago, IL (US); Bruce Bach, North Chicago, IL (US)

(73) Assignee: AbbVie Inc., North Chicago, IL (US)

(21) Appl. No.: 16/626,769

(22) PCT Filed: Jun. 26, 2018
(86) PCT No.: PCT/US2018/039453

§ 371 Date: Dec. 26, 2019

Related U.S. Application Data
(60) Provisional application No. 62/524,711, filed on Jun. 26, 2017.

(51) Int. Cl. A61K 31/4184; A61P 35/00; A61K 31/555; A61K 31/337

(52) U.S. Cl. CPC A61K 31/4184; A61K 31/337; A61K 31/555; A61P 35/00

(57) ABSTRACT

The present invention provides methods of treating non-small cell lung cancer in a patient comprising administering to the patient an effective amount of a PARP inhibitor, wherein the patient is Lung Subtyping Panel (LSP) positive.

### CROSS-REFERENCE TO RELATED APPLICATIONS

This application claims priority to U.S. Provisional Application No. 62/524,711, filed on Jun. 26, 2017, which is incorporated herein by reference in its entirety.

### BACKGROUND

Lung cancer accounts for more deaths than any other cancer in both men and women. An estimated 159,260 deaths, accounting for about 27% of all cancer deaths, are expected to occur in the United States in 2014 (American Cancer Society. Cancer Facts & Figures 2014. Atlanta: American Cancer Society; 2014). Lung cancer is broadly classified into two types: non-small cell lung cancer and small cell lung cancer. Non-small cell lung cancer (NSCLC) comprises 80-85% of lung cancer cases in the United States. NSCLC comprises three major types: (i) Squamous cell carcinoma, which begins in squamous cells, that are thin, flat cells that look like fish scales. Squamous cell carcinoma is also called epidermoid carcinoma; (ii) Large cell carcinoma, which begins in several types of large lung cells; and (iii) Adenocarcinoma, which begins in the cells that line the alveoli of the lung and make substances such as mucus. Other less common types of NSCLC include pleomorphic carcinoma, carcinoid tumor and unclassified carcinoma.

The diagnosis of non-small cell lung cancer histologic subtype is the current gold standard for appropriate selection of chemotherapy. Recent studies showed that the histologic types of non-small cell lung cancer differ not only in their morphologic features but also in their genetic sequence mutations and expression patterns. Several studies have confirmed the use of gene expression analysis to determine the histologic subtype of lung cancer. For example, gene expression analysis using quantitative real-time PCR (qRT-PCR) for 57 genes expressed in lung cancer has been used to classify adenocarcinoma, squamous cell carcinoma, and neuroendocrine (NE, small cell lung cancer and carcinoid) subtypes of lung cancer. However, the challenges in reconciling gene expression analysis across various histologic subtypes of NSCLC and applying the results to personalize cancer treatment are formidable.

Therefore, there is a continuing need in the art to identify new classification systems that are accurate and reliable for selecting cancer therapies that target a specific patient individual or population.

### SUMMARY OF THE INVENTION

The present invention provides methods of treating non-small cell lung cancer in a patient comprising the step of administering to the patient an effective amount of a PARP inhibitor, wherein the patient is Lung Subtyping Panel (LSP) positive.

In one embodiment, the LSP positive patient is tested to be positive for one or more LSP markers prior to the administration of the PARP inhibitor.

In one embodiment, the LSP markers comprise at least one of NKX2-1, DSC3 and HPN, or additionally at least one of HNF1B and ALDH3B1, or additionally at least one markers selected from a group consisting of CDH5, DOK1, PECAM1, HYAL2 and CLEC3B, or additionally at least one of MGRN1 and ME3, or additionally at least one markers selected from a group consisting of NKX2-1, DSC3, HPN, HNF1B, ALDH3B1, CDH5, DOK1, PECAM1, HYAL2, CLEC3B, MGRN1 and ME3, or additionally at least one genes selected from a group consisting of ABCC5, ACVR1, ANTXR1, BMP7, CACNB1, CAPG, CBX1, CDKN2C, CFL1, CHGA, CIB1, CYB5B, EEF1A1, FEN1, FOXH1, GJB5, HOXD1, ICA1, ICAM5, INSM1, ITGA6, LGALS3, LIPE, LRP10, MAPRE3, MYBPH, MYO7A, NFIL3, PAICS, PAK1, PIK3C2A, PLEKHA6, PSMD14, RPL10, RPL28, RPL37A, SCD5, SFN, SIAH2, SNAP91, STMN1, TCP1, TFAP2A, TRIM29 and TUBA4A.

In one embodiment, the PARP inhibitor is a PAPR-1 inhibitor, a PARP-2 inhibitor, or a PARP-1/2 inhibitor.

In one embodiment, the PARP inhibitor is selected from a group consisting of veliparib, niraparib, olaparib, rucaparib and talazoparib. In a preferred embodiment, the PARP inhibitor is veliparib.

In one embodiment, the PARP inhibitor is administered in combination with one or more other anti-tumor agents. In one embodiment, the anti-tumor agent is carboplatin, paclitaxel, or both.

In one embodiment, the NSCLC cancer is squamous.

In one embodiment, the NSCLC cancer is non-squamous.

The present invention also provides methods of testing, selecting or predicting an LSP positive patient for PARP inhibitor treatment, and kits for such test, selection or prediction.

### BRIEF DESCRIPTION OF THE DRAWINGS

FIG. 1 shows veliparib benefit observed in signature subgroup in both M10-898 and M11-089 studies.

FIG. 2 shows increased Overall Survival (OS) observed in LSP positive NSCLC patients treated with veliparib.

FIG. 3 shows cross-validated prediction error curve along with the number of LSP markers.

FIG. 4 is a diagram of the stemness score of TCGA lung cancer adenocarcinoma (LUAD), TCGA lung cancer squamous cell carcinoma (LUSC), M11-089 and M14-359 clinical studies.

FIG. 5 shows that veliparib treatment provides a benefit to subgroup of patients which were determined by a stemness score of at least 0.5 or less than 0.5.

FIG. 6 is a diagram depicting the correlation of a LSP+ status and TP53 inactivation signature score.

FIG. 7 shows the correlation between LSP+ status and proliferation index.

FIG. 8 depicts the comparison of histological analysis versus LSP status. LSP status defines a specific subpopulation within each of histological subtypes of tumors that benefit from PARP inhibitor treatment.

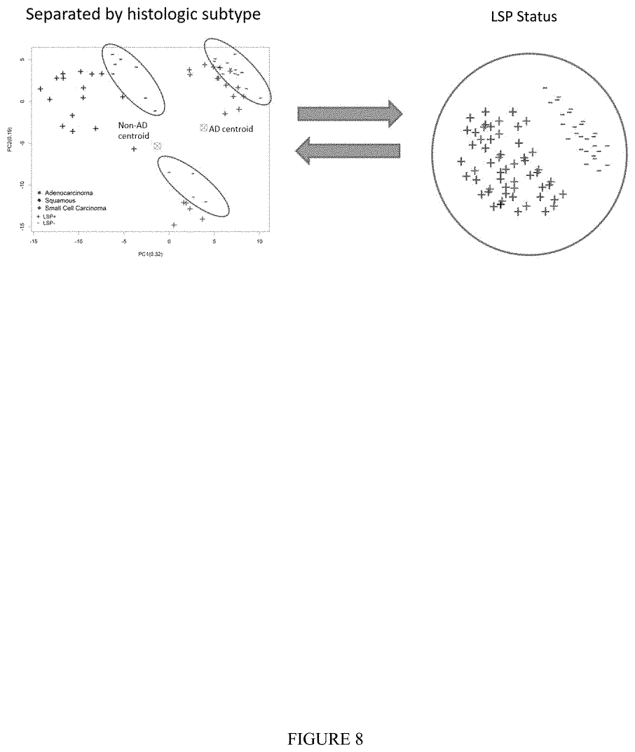

### DESCRIPTION OF THE INVENTION

The present invention is based on unexpected discoveries that gene expressions of Lung Subtyping Panel markers can be used to identify a specific NSCLC patient population that is likely to have a favorable response to PARP inhibitor treatment, independent of their histologic tumor classifications.

Accordingly, one aspect of the present invention is a method of treating non-small cell lung cancer in a patient who is Lung Subtyping Panel (LSP) positive comprising a step of administering to the patient an effective amount of a PARP inhibitor.

The term “treatment” can be characterized by at least one of the following: (a) the reducing, slowing or inhibiting the spread of cancer and cancer cells, including slowing, inhibiting or reducing the growth of metastatic cancer cells; (b) preventing the further growth of tumors; (c) reducing or preventing the metastasis of cancer cells within a subject; or (d) reducing or ameliorating at least one symptom of cancer within the subject. The methods of “treatment” use administration to a patient of a PARP inhibitor as provided herein, for example, in patient having cancer in order to prevent, cure, delay, reduce the severity of, or ameliorate one or more symptoms of the cancer or in order to prolong the survival of a patient beyond that expected in the absence of the treatment.

The term “patient” refers to a human subject.

The term “PARP inhibitors” refer to compounds or agents that inhibit or retard the activation of, or enzymatic activity of, poly-ADP ribose polymerase (PARP). The PARP inhibitors can be PARP-1 inhibitors, PARP-2 inhibitors, or PARP-1 and -2 inhibitors.

The term “Lung Subtyping Panel” or “LSP” refers to tumor genetic signatures or molecular behavior profiles that can be used for an objective means of classifying lung tumors, more importantly, for evaluation of a NSCLC patient's response to cancer treatment with PARP inhibitors.

As demonstrated in the Examples, the LSP positive or LSP+ subpopulation described herein refers to a subgroup of lung cancer patients that may benefit from the treatment with a PARP inhibitor, regardless of their histologic classification. It is the unique gene signature profile of the LSP+ patient that provides the benefit of treatment using a PARP inhibitor.

The LSP profile described herein to characterize a patient's tumor as LSP+ is used synonymously with other names or acronyms, including, but not limited to, for example, lung prognostic profile (LPP), LPP-52, lung molecular prognostic signature (LMPS), lung molecular prognostic profile, lung molecular signature (LMS), LMS-52, lung-52, lung prognosis 52 (LP-52) and the like. These names are used interchangeably.

In some embodiments, a LSP+ NSCLC patient is characterized as a NSCLC patient who has poor prognosis, high proliferation index, high stemness score, increased P53 inactivation, or preferably, positive or predefined expression pattern of at least one of LSP panel markers.

Proliferation index is a measure of the number of cells in a tumor that are dividing or proliferating. Several technologies have been developed to evaluate the proliferation index in tumor samples, for example, including mitotic indexing, thymidine-labeling index, bromodeoxyuridine assay, the determination of fraction of cells in various phases of cell cycle, and the immunohistochemical evaluation of cell cycle-associated proteins.

In one embodiment, a LSP+ patient of the present invention is a NSCLC patient whose tumor has a proliferation index higher than zero (0), for example, based on the proliferation index used as a supplement for PAM50 signature.

Stemness is defined as the potential for self-renewal and differentiation from the cell of origin, for example, normal stem cells that possess the ability to give rise to all cell types in the adult organism. Cancer progression involves the gradual loss of a differentiated phenotype and the acquisition of stem-cell-like features. To evaluate degrees of stemness of a tumor, or stemness score, a predictive model using one-class logistic regression (OCLR) on pluripotent stem cell samples can be built. To score a new sample, spearman correlation is computed between model's weight vector and the new sample expression profile.

In some embodiments, stemness score can be determined based on mDNAsi or mRNAsi using OCLR by combining (1) supervised classification between ESCs/iPSCs and their progenies, (2) stem cell signatures associated with pluripotency-specific genomic enhancer elements based on ChromHMM from Roadmap, and (3) ELMER, which uses DNA methylation to identify enhancer elements and correlates their state with the expression of nearby genes.

In one embodiment, a LSP+ patient of the present invention has a NSCLC tumor having a stemness score of at least 0.5.

In one specific embodiment, a LSP+ patient of the present invention has a NSCLC tumor having a stemness mRNAsi score of at least 0.5.

TP53 is known tumor suppressor gene, which is frequently inactivated by mutations or deletion in human tumors. For example, the loss of TP53 has been observed in over 1/3 of DNA damage repair (DDR) genes, indicating alterated DNA damage repair pathways.

In one embodiment, a LSP+ patient of the present invention is a NSCLC patient whose TP53 gene has been inactivated at least 50% (TP53 deficiency) as compared to a patient who is not LSP+.

In some embodiments, a panel of LSP markers listed in Table 1 is used to characterize a patient's tumor as LSP+, and the LSP markers of the present invention include any one of those genes or proteins thereof that have a selectively expression pattern in lung cancer cells:

TABLE 1
LSP Markers

| Gene Symbol | Entrez Gene Name |
|---|---|
| ABCC5 | ATP binding cassette subfamily C member 5 |
| ACVR1 | activin A receptor type 1 |
| ALDH3B1 | aldehyde dehydrogenase 3 family member B1 |
| ANTXR1 | anthrax toxin receptor 1 |
| BMP7 | bone morphogenetic protein 7 |
| CACNB1 | calcium voltage-gated channel auxiliary subunit beta 1 |
| CAPG | capping actin protein, gelsolin like |
| CBX1 | chromobox 1 |
| CDH5 | cadherin 5 |
| CDKN2C | cyclin dependent kinase inhibitor 2C |
| CFL1 | cofilin 1 |
| CHGA | chromogranin A |
| CIB1 | calcium and integrin binding 1 |
| CLEC3B | C-type lectin domain family 3 member B |
| CYB5B | cytochrome b5 type B |
| DOK1 | docking protein 1 |
| DSC3 | desmocollin 3 |
| EEF1A1 | eukaryotic translation elongation factor 1 alpha 1 |
| FEN1 | flap structure-specific endonuclease 1 |
| FOXH1 | forkhead box H1 |
| GJB5 | gap junction protein beta 5 |
| HNF1B | HNF1 homeobox B |
| HOXD1 | homeobox D1 |
| HPN | hepsin |
| HYAL2 | hyaluronoglucosaminidase 2 |
| ICA1 | islet cell autoantigen 1 |
| ICAM5 | intercellular adhesion molecule 5 |
| INSM1 | INSM transcriptional repressor 1 |
| ITGA6 | integrin subunit alpha 6 |
| LGALS3 | galectin 3 |
| LIPE | lipase E, hormone sensitive type |
| LRP10 | LDL receptor related protein 10 |
| MAPRE3 | microtubule associated protein RP/EB family member 3 |
| ME3 | malic enzyme 3 |
| MGRN1 | mahogunin ring finger 1 |
| MYBPH | myosin binding protein H |
| MYO7A | myosin VIIA |
| NFIL3 | nuclear factor, interleukin 3 regulated |
| NKX2-1 | NK2 homeobox 1 |
| PAICS | phosphoribosylaminoimidazole carboxylase and |
| | phosphoribosylaminoimidazolesuccinocarboxamide |
| | synthase |
| PAK1 | p21 (RAC1) activated kinase 1 |
| PECAM1 | platelet and endothelial cell adhesion molecule 1 |
| PIK3C2A | phosphatidylinositol-4-phosphate 3-kinase catalytic |
| | subunit type 2 alpha |
| PLEKHA6 | pleckstrin homology domain containing A6 |
| PSMD14 | proteasome 26S subunit, non-ATPase 14 |
| RPL10 | ribosomal protein L10 |
| RPL28 | ribosomal protein L28 |
| RPL37A | ribosomal protein L37a |
| SCD5 | stearoyl-CoA desaturase 5 |
| SFN | stratifin |
| SIAH2 | siah E3 ubiquitin protein ligase 2 |
| SNAP91 | synaptosome associated protein 91 |
| STMN1 | stathmin 1 |
| TCP1 | t-complex 1 |
| TFAP2A | transcription factor AP-2 alpha |
| TRIM29 | tripartite motif containing 29 |
| TUBA4A | tubulin alpha 4a |

In some embodiments, the LSP markers can be placed in four tiers based on their respective predictive power in determining LSP positivity of a NSCLC patient.

The first-tier markers include NKX2-1, DSC3 and HPN.

The second-tier markers include HNF1B and ALDH3B1.

The third-tier includes markers CDH5, DOK1, PECAM1, HYAL2, CLEC3B, MGRN1 and ME3.

The fourth-tier markers include ABCC5, ACVR1, ANTXR1, BMP7, CACNB1, CAPG, CBX1, CDKN2C, CFL1, CHGA, CIB1, CYB5B, EEF1A1, FEN1, FOXH1, GJB5, HOXD1, ICA1, ICAM5, INSM1, ITGA6, LGALS3, LIPE, LRP10, MAPRE3, MYBPH, MYO7A, NFIL3, PAICS, PAK1, PIK3C2A, PLEKHA6, PSMD14, RPL10, RPL28, RPL37A, SCD5, SFN, SIAH2, SNAP91, STMN1, TCP1, TFAP2A, TRIM29 and TUBA4A.

Any of LSP markers from each of the four tiers can be used alone or in combination with other markers in the same tier or in combination with markers from different tier or tiers, as long as the markers or the combination of the markers can provide sufficient predicative power or confidence for determining the LSP positivity.

In one embodiment, the LSP markers of the present invention comprise at least one of the first-tier markers.

In one embodiment, the LSP markers comprise at least one of the first-tier markers and at least one of the second-tier markers.

In one embodiment, the LSP markers comprise at least one of the first-tier markers and at least one of third-tire markers.

In one embodiment, the LSP markers comprise at least one of the first-tier markers and at least one of the fourth-tire markers.

In one embodiment, the LSP markers comprise at least one of the first-tier markers, at least one of the second-tier markers, and at least one of the third-tier markers.

In one embodiment, the LSP markers comprise at least one of the first-tier markers, at least one of the second-tier markers, and at least one of the fourth-tier markers.

In one embodiment, the LSP markers comprise at least one of the first-tier markers, at least one of the third-tier markers, and at least one of the fourth-tier markers.

In one embodiment, the LSP markers comprise at least one of the first-tier markers, at least one of the second-tier markers, at least one of the third-tier markers, and at least one of the fourth-tier markers.

In one embodiment, the LSP markers of the invention comprise at least one of NKX2-1, DSC3 and HPN, preferably at least two of NKX2-1, DSC3 and HPN, and more preferably all NKX2-1, DSC3 and HPN.

In one embodiment, in addition to NKX2-1, DSC3 and HPN, the LSP markers comprise either HNF1B or ALDH3B1, or preferably both of HNF1B and ALDH3B1.

In one embodiment, in addition to NKX2-1, DSC3, HPN, HNF1B and ALDH3B1, the LSP markers comprise at least one markers selected from a group consisting of CDH5, DOK1, PECAM1, HYAL2 and CLEC3B.

In one embodiment, in addition to NKX2-1, DSC3, HPN, HNF1B, ALDH3B1, CDH5, DOK1, PECAM1, HYAL2 and CLEC3B, the LSP markers comprise MGRN1 or ME3, or both.

In one embodiment, the LSP markers comprise at least one markers selected from a group consisting of NKX2-1, DSC3, HPN, HNF1B, ALDH3B1, CDH5, DOK1, PECAM1, HYAL2, CLEC3B, MGRN1 and ME3.

In one embodiment, the LSP markers comprise NKX2-1, DSC3, HPN, HNF1B, ALDH3B1, CDH5, DOK1, PECAM1, HYAL2, CLEC3B, MGRN1 and ME3.

In one embodiment, one or more of ABCC5, ACVR1, ANTXR1, BMP7, CACNB1, CAPG, CBX1, CDKN2C, CFL1, CHGA, CIB1, CYB5B, EEF1A1, FEN1, FOXH1, GJB5, HOXD1, ICA1, ICAM5, INSM1, ITGA6, LGALS3, LIPE, LRP10, MAPRE3, MYBPH, MYO7A, NFIL3, PAICS, PAK1, PIK3C2A, PLEKHA6, PSMD14, RPL10, RPL28, RPL37A, SCD5, SFN, SIAH2, SNAP91, STMN1, TCP1, TFAP2A, TRIM29 and TUBA4A is added to the list of LSP markers described in the embodiments above.

When used above-mentioned LSP markers, a “LSP positive” patient means a patient who expresses one or more of the LSP markers or a combination of the LSP markers at a probability level higher than a “reference level.” Extent of upregulated or downregulated expressions of the LSP markers relative to the reference level is indicative of an increased or decreased probability that the patient would be sensitive to PARP inhibitors therefore would be benefit from such treatment.

The term “reference level” refers to a specific value or dataset that can be used to classify the value (e.g. expression level) or reference expression profile obtained from a test sample associated with a desired outcome. It can be established in various ways and may be an absolute or relative amount.

In one embodiment, a reference level of LSP markers for a NSCLC status, phenotypes or histological types, or lack thereof, may be determined by measuring levels of selected LSP markers in samples from one patient or a group of patients, and such reference levels may be tailored to specific patient populations.

In one embodiment, a reference level of the LSP marks may be associated with NSCL histological subtype-matched and based on quantitative analysis of LSP markers so that comparisons may be made between samples from patients with a certain NSCLC histological subtype and samples from patients with a different NSCLC histological subtype. Examples of NSCLC histological subtypes include, but not limited to, neuroendocrine (NE) such as small cell carcinoma, large cell carcinoma, carcinoid tumor, squamous (SQ) and non-squamous NSCLC (NSQ) such as adenocarcinoma (AD).

In one embodiment, a reference level of the LSP markers is derived from a set of samples with a LSP gene expression profile that is known to be associated with a histological subtype of NSCLC (a training set). The training set is then tested against a test sample, and comparison between the training set and the test sample allows the quantitative description of the multivariate boundaries that characterize and separate each class, for example, each class of NSCLC in terms of its LSP biomarker expression profile or subtypes of NSCLC. A reference level can also be based on confidence limits for any predictions, for example, a level of probability to be placed on the goodness of fit of the LSP markers in the test sample as compared to those in the training set, or be derived from a centroid based method or other types of statistical algorithm methods, in which one training set or multiple of training sets are used to determine LSP marker profile of a test sample from a patient.

For example, a reference level can be developed based on three training sets—one training set for the expression profile of a set of LSP markers from adenocarcinoma samples, one training set for the expression profile of the same set of LSP markers from squamous samples, and one training set for the expression profile of the same set of LSP markers from neuroendocrine samples. A test sample is obtained from a patient and its expression profile of the same set of LSP markers is detected then compared to two training set through a statistical algorithm so a correlation is established between the three expression profiles. The LSP positivity or classification of the test sample can be determined based on the statistical algorithm.

Besides the markers listed in Table 1, KRT5 or K167 can also be used as surrogate markers to determine if a patient is LSP positive or not. KRT5 and K167 are well-established markers for squamous lineage tumors or proliferation process, respectively. The present invention demonstrates that expression of either or both of KRT5 and K167 correlates LSP positivity.

In some embodiments, LSP positive patients have squamous NSCLC.

In some embodiments, LSP positive patients have non-squamous NSCLC.

In some embodiments, LSP positive patients are not positive for one of driver mutations including, but not limited to, EGFR, ALK and ROS.

In some embodiments, LSP positive patients have low PD-L1 expression. In preferred embodiments, LSP positive patients have tumor proportion score (TPS) of lower than 50% for PD-L1.

In some embodiments, LSP positive patients have advanced NSCLC.

In some embodiments, LSP positive patients have metastatic NSCLC.

Once a patient is identified to be LSP positive, the patient can be given an effective amount of PARP inhibitor treatment. The PARP inhibitor treatment can be monotherapy where a PARP inhibitor is alone or be a combination therapy where the PARP inhibitor is administered with other anti-tumor agents.

The term “effective amount” refers to the amount sufficient to induce a desired biological, pharmacological, or therapeutic outcome in a subject at a reasonable benefit/risk ratio applicable to medical treatments, more particularly, to PARP inhibitor treatment.

In one embodiment, a PARP inhibitor is administered to a LSP positive NSCLC patient as monotherapy.

In one embodiment, a PARP inhibitor is administered to a LSP positive NSCLC patient in combination with standard of care treatment for NSCLC.

In one embodiment, a PARP inhibitor is administered to a LSP positive NSCLC patient in combination with one or more chemotherapeutic agents.

In one embodiment, a PARP inhibitor is administered to a LSP positive NSCLC patient in combination with carboplatin.

In one embodiment, a PARP inhibitor is administered to a LSP positive NSCLC patient in combination with paclitaxel.

In one embodiment, a PARP inhibitor is administered to a LSP positive NSCLC patient in combination with carboplatin and paclitaxel.

In one embodiment, the PARP inhibitor is a PAPR-1 inhibitor, a PARP-2 inhibitor, or a PARP-1/2 inhibitor.

In one embodiment, the PARP inhibitor is selected from a group consisting of veliparib, niraparib, olaparib, rucaparib and talazoparib. In a preferred embodiment, the PARP inhibitor is veliparib.

In a preferred embodiment, the PARP inhibitor for treating a LSP positive patient is veliparib, optionally in combination with carboplatin, paclitaxel, or both.

In a more preferred embodiment, the PARP inhibitor for treating a LSP positive patient is veliparib, wherein the veliparib is used in combination with carboplatin and paclitaxel.

In one embodiment, the effective amount of veliparib is in the range of 20 to 600 mg or in the range of 60 to 400 mg. In a further embodiment of the invention, the effective amount of veliparib is about 30 mg, 50 mg, 80 mg, 100 mg, 120 mg, 150 mg, 200 mg, 240 mg, or 300 mg. In one embodiment, the dose is administered multiple times per day. In one embodiment, veliparib is administered once a day or twice a day. In one embodiment, veliparib is administered twice a day.

In one preferred embodiment, veliparib is administered at a dose of 120 mg, twice a day.

After receiving the above-mentioned treatment, the LSP+ patients will be effectively treated. The term “effective treatment” refers to treatment producing a beneficial effect, e.g., amelioration of at least one symptom of NSCLC. A beneficial effect can take the form of an improvement over baseline, i.e., an improvement over a measurement or observation made prior to initiation of therapy according to the method. A beneficial effect can also take the form of reducing, inhibiting or preventing growth or metastasis of the cancer cells or invasiveness of the cancer cells or metastasis or reducing, alleviating, inhibiting or preventing at least one symptoms of the cancer. Such effective treatment may, e.g., reduce patient pain, reduce the size or number of cancer cells, may reduce or prevent metastasis of a cancer cell, or may slow metastatic cell growth.

In some embodiments, the effective treatment is measured by overall survival (OS) or progression-free survival (PFS).

Time to death (overall survival, OS) for a given subject was defined as the number of days from the date that the subject was randomized to the date of the subject's death. All events of death were included, regardless of whether the event occurred while the subject was still taking veliparib/placebo or had previously discontinued veliparib/placebo. If a subject did not die during the study, then the data were censored at the date when the subject was last known to be alive.

PFS was defined as the number of days from the date that the subject was randomized to the date the subject experienced an event of disease progression (as determined by a central imaging center) or to the date of death (all causes of mortality) if disease progression was not reached. All events of disease progression (as determined by the central imaging center) were included, regardless of whether the event occurred while the subject was still taking veliparib/placebo or had previously discontinued veliparib/placebo. However, if a disease progression event occurred after a subject missed two or more consecutive disease progression assessments; the subject was censored at the last disease progression assessment prior to the missing disease progression assessments. All events of death were included for subjects who had not experienced disease progression, provided the death occurred within 42 days of the last disease assessment. If the subject did not have an event of disease progression (as determined by the central imaging center) nor had the subject died, the subject's data was censored at the date of the subject's last disease assessment.

In one embodiment, the treatment of veliparib in combination with carboplatin and paclitaxel increases overall survival (OS) of LSP+ patients by at least one month as compared to the LSP+ patients who receive the chemotherapy alone. In one embodiment, the OS is increased by at least two months. In one embodiment, the OS is increased by at least three months. In one embodiment, the OS is increased by at least four months. In one embodiment, the OS is increased by at least five months. In one embodiment, the OS is increased by at least 6 months. In one embodiment, the OS is increased by at least 7 months.

In one embodiment, the treatment of veliparib in combination with carboplatin and paclitaxel increases progression-free survival (PFS) of LSP+ patients by at least one month as compared to the LSP+ patients who receive the chemotherapy alone. In one embodiment, the PFS is increased by at least 1.5 months. In one embodiment, the PFS is increased by at least 2 months. In one embodiment, the PFS is increased by at least 2.5 months. In one embodiment, the PFS is increased by at least 3 months. In one embodiment, the PFS is increased by at least 3.5 months. In one embodiment, the PFS is increased by at least 4 months.

The LSP marker panel may be used in methods of evaluating if a patient would benefit from PARP inhibitor treatment. In some embodiments, methods of detecting the presence of one or more markers in the LSP panel of the invention in a biological sample involves obtaining a biological sample (e.g. tumor sample) from a test subject and contacting the biological sample with a compound or an agent capable of detecting the nucleic acid (mRNA, genomic DNA or cDNA) of the marker within the sample.

In another aspect, the present invention provides a method of testing a LSP positive NSCLC patient for PARP inhibitor treatment, wherein the method comprises the step:

(a) determining the expression of one or more of LSP markers in a test sample from the patient.

In one embodiment, the method above additionally comprises the step:

(b) comparing the expression of one or more of the LSP markers in the test sample with a reference level, wherein a difference in the expression between the test sample and the reference level is indicative of the LSP positivity of the patient.

In one embodiment, the method above further comprises the step:

(c) administered an effective amount of a PARP inhibitor to the patient that is LSP positive.

In another aspect, the present invention provides a method of predicting a NSCLC patient suitable for PARP inhibitor, wherein the method comprises the step:

(a) determining the expression of one or more of LSP markers in a test sample from the patient.

In one embodiment, the method above additionally comprises the step:

(b) comparing the expression of one or more of the LSP markers in the test sample with a reference level, wherein a difference in the expression between the test sample and the reference level is indicative of the LSP positivity of the patient.

In one embodiment, the method above further comprises the step:

(c) administered an effective amount of a PARP inhibitor to the patient that is LSP positive.

In another aspect, the present invention provides a method of selecting a therapy for a NSCLC patient, wherein the method comprises the steps:

(a) determining the expression of one or more of LSP markers in a test sample from the patient.

(b) comparing the expression of one or more of the LSP markers in the test sample with a reference level, wherein a difference in the expression between the test sample and the reference level is indicative of the LSP positivity of the patient.

(c) selecting PARP inhibitor therapy for the patient that is LSP positive.

In another aspect, the present invention provides a method of predicting a NSCLC patient suitable for PARP inhibitor therapy, wherein the method comprises the steps:

(a) determining the expression of one or more of LSP markers in a test sample from the patient.

(b) comparing the expression of one or more of the LSP markers in the test sample with a reference level, wherein a difference in the expression between the test sample and the reference level is indicative of the LSP positivity of the patient,

wherein the patient is suitable for PARP inhibitor therapy if the patient is LSP positive.

As noted herein, the term “determining the expression of LSP biomarkers” refers to determining or quantifying RNA or proteins expressed by the LSP biomarkers. The term “RNA” includes mRNA transcripts, and/or specific spliced variants of mRNA. The terms “RNA product of the biomarker,” “biomarker RNA,” or “target RNA” as used herein refers to RNA transcripts transcribed from the biomarkers and/or specific spliced variants. In the case of “protein”, it refers to proteins translated from the RNA transcripts transcribed from the biomarkers. The term “protein product of the biomarker” or “biomarker protein” refers to proteins translated from RNA products of the biomarkers.

A person skilled in the art will appreciate that various methods can be used to detect or quantify the level of RNA products of the biomarkers within a sample, including arrays, such as microarrays, RT-PCR (including quantitative PCR), nuclease protection assays and Northern blot analyses and next-generation sequencing. Any analytical procedure capable of permitting specific and quantifiable (or semi-quantifiable) detection of the LSP markers may be used in the methods herein presented, such as the microarray methods, and methods known to those skilled in the art.

In one embodiment, the biomarker expression levels are determined using arrays, such as microarrays, RT-PCR, quantitative RT-PCR, nuclease protection assays or Northern blot analyses and next-generation sequencing.

In one preferred embodiment, the biomarker expression levels are determined by using quantitative RT-PCR. The first step is the (RT-qPCR) isolation of mRNA from a test sample. The starting material is typically total RNA isolated from human tumors or tumor cell lines. General methods for mRNA extraction are well known in the art. For example, RNA isolation can be performed using purification kit, buffer set and protease from commercial manufacturers, such as Qiagen. The second step is the detection of the selected LSP marker RNA, typically by using a forward and reverse primer for the LSP marker genes. Kits for RT-PCR analysis are commercially available.

In some embodiments, quantitation of biomarker RNA expression levels requires assumptions to be made about the total RNA per cell and the extent of sample loss during sample preparation. In some embodiments, the addressable array comprises DNA probes for each of the selected LSP marker genes and, optionally, one, two, three, four, or five housekeeping genes. In one embodiment, the housekeeping genes include, but not limited to, any one or more of CFL1, EEF1A1, RPL10, RPL28 and RPL37A.

In some embodiments, kits for carrying out the methods described herein are provided. The kits provided may contain the necessary components with which to carry out one or more of the above-noted methods. In one embodiment, a kit for treating cancer is provided. In another embodiment, a kit for evaluating a NSCLC patient as LSP positive or for identifying a LSP positive patient suitable for PARP inhibitor treatment are provided. In one such embodiment, the kit comprises a panel able to detect at least one LSP marker to identify a LSP positive patient. In some embodiments, the kit further includes a PARP inhibitor capable of being administered to the LSP positive patient. In one embodiment, the panel comprises at least 12 LSP markers for determining if a LSP positive patient, and in some examples, further contains a PARP inhibitor to treat the LSP positive patient.

In yet another aspect, the invention also provides a kit used to test if a NSCLC patient is LSP positive or to identify a LSP positive patient suitable for PARP inhibitor treatment, wherein the kit comprises detection agents that can detect the expression of one or more of LSP markers as described herein.

In yet another aspect, the invention provides a kit for selecting PARP inhibitor therapy for a NSCLC patient, comprising detection agents that can detect the expression of one or more of LSP markers as described herein.

In some embodiments, the kits of the present invention for use in the RT-PCR methods described herein comprise one or more target RNA-specific probes and one or more primers for reverse transcription of target RNAs of LSP markers or amplification of cDNA reverse transcribed therefrom.

In some embodiments, the kits of the present invention for use in the RT-PCR methods additionally comprise primers that are specific to one or more housekeeping genes for use in normalizing the quantities of target RNAs of LSP markers. Such probes (and primers) include those that are specific for one or more products of housekeeping genes selected from CFL1, EEF1A1, RPL10, RPL28 and RPL37A.

In some embodiments, the kits can also include a reference level. The kits may additionally include instructions for using the reference level.

The above disclosure generally describes the present invention. A more complete understanding can be obtained by reference to the following specific examples. These examples are described solely for illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

The following non-limiting example is illustrative of the present invention:

### EXAMPLES

### Example 1

Clinical Protocol Design

The objective of the clinical study was to assess if treatment with veliparib plus standard chemotherapy could result in improved survival in LSP positive subjects with metastatic or advanced NSCLC. The standard chemotherapies include, but not limited to, platinum doublet chemotherapy (carboplatin/paclitaxel, cisplatin/pemetrexed, or carboplatin/pemetrexed). The recommended chemotherapy to be used in combination with veliparib was carboplatin and/or paclitaxel.

Subjects are randomized in a 1:1 ratio to a maximum of 6 cycles of carboplatin/paclitaxel plus 120 mg BID of veliparib or a maximum of 6 cycles of Investigator's choice of platinum doublet chemotherapy (carboplatin/paclitaxel, cisplatin/pemetrexed, or carboplatin/pemetrexed), unless treatment is discontinued for toxicity or cancer progression. Investigators may elect to administer maintenance pemetrexed regardless of which therapy their subjects are randomized to receive.

Subjects LSP status (positive or negative) are determined from tissue samples obtained. Subjects randomized to receive veliparib would begin oral veliparib dosing 2 days prior to the start of the carboplatin/paclitaxel infusion on C1D-2 and will continue twice a day (BID) through C1D5 (7 consecutive days). Subjects randomized to receive carboplatin/paclitaxel/veliparib would receive carboplatin (AUC 6 mg/mL•min) and paclitaxel (200 mg/m2) IV infusion starting on Day 1 of each cycle. Subjects would receive a maximum of 6 cycles of treatment, unless toxicity requires cessation of therapy, or radiographic progression occurs prior to completing 6 cycles. Carboplatin/paclitaxel plus veliparib may be delayed or dose-modified due to toxicity.

Subjects randomized to receive Investigator's choice of platinum doublet therapy would receive therapy on Day 1 of each cycle. Subjects would receive a maximum of 6 cycles of treatment, unless toxicity requires cessation of therapy, or radiographic progression occurs prior to completing 6 cycles. Platinum doublet therapy may be delayed or dose-modified due to toxicity. Dose delays and modification would be at the discretion of the Investigator per local standard practice.

Recommended Dose Regimen:

Veliparib: 120 mg BID Days—2 through 5 of 21-day cycle

Carboplatin: Day 1 of 21-day cycle, AUC 6 mg/mL•min, intravenous

Paclitaxel: Day 1 of 21-day cycle, 200 mg/m², intravenous

### Example 2

Assay

By measuring expression of component genes (Table 1) and applying a classification algorithm one can determine if a tumor sample is LSP positive or LSP negative. LSP status is a predictive indicator in patients having at least one type of cancer, including cancers characterized by the presence of a tumor, such as a lung cancer tumor, particularly in the context of a therapeutic regimen involving a PARP binding agent in combination with DNA damaging chemotherapy including carboplatin and paclitaxel.

The specific assay steps starting with tumor biopsy materials through to LSP status determination are outlined below. This can be further segregated into the processes to measure expression of the component genes and the assignment of LSP status using a classification algorithm based on component gene expression.

The assignment of LSP status using the classification algorithm was broken down into utilization of the classification algorithm to apply LSP status for new samples and construction of the classification algorithm.

Measurement of Component Gene Expression

Tumor dissection and nucleic acid isolation: Tumor RNA was prepared in a manner compatible with the analytical technique ultimately used to measure expression of component genes. RNA isolation was a prerequisite of the methodology. Tumor DNA/RNA was obtained by macrodissecting tumor area to ensure >50% tumor content. RNA and DNA were isolated using an AllPrep kit (Qiagen) according to the manufacturer's protocol. Other RNA isolation methods exist and would produce RNA suitable for various analytical methods to measure component gene expression. Those additional RNA isolation methods likely can be used in lieu of AllPrep. Additionally, some analytical methods to measure component gene expression do not require purified RNA as an input (including ribonuclease protection). RNA isolation may be omitted if the analytical method used to measure component gene expression is not dependent upon having purified RNA as a starting input.

RNAseq Library Preparation and Sequencing: The analytical technique used to measure component gene expression was RNA sequencing (RNAseq). The specific methods below were used although alternative methods could be substituted.

The integrity assessment of isolated RNA was performed using an Agilent bioanalyzer and quantitated using picogreen. Library preparation was performed with 1-50 ng of total RNA. Double-stranded-complementary DNA (ds-cDNA) was prepared using the SeqPlex RNA Amplification Kit (Sigma) per manufacturer's protocol. Complementary DNA (cDNA) was blunt ended, had an A base added to the 3′ ends, and then had Illumina sequencing adapters ligated to the ends. Ligated fragments were then amplified for 12 cycles using primers incorporating unique index tags. Fragments were sequenced on an Illumina HiSeq-2500 or HiSeq-3000 using single reads extending 50 bases. 25-30M reads per library are targeted.

RNA-seq Data Acquisition, Quality Control, and Processing: RNA sequencing reads were aligned to the Ensembl release 76 assembly with STAR version 2.0.4b. Gene counts were derived from the number of uniquely aligned unambiguous reads by Subread:featureCount version 1.4.5. Transcript counts were produced by Sailfish version 0.6.3. Sequencing performance was assessed for total number of aligned reads, total number of uniquely aligned reads, genes and transcripts detected, ribosomal fraction known junction saturation, and read distribution over known gene models with RSeQC version 2.3.

Assignment of LSP status using component gene expression: The process of distilling the expression of the LSP classifier component genes into an LSP status utilizes a classification algorithm. The nearest shrunken centroid method was used by AbbVie for construction of the classification algorithm. To obtain the LSP classification for M11-089 and M14-359 samples, batch-wise and pathology-wise z-score normalization was applied to the log transformed RPKM RNAseq data from three batches of M11-089 and one batch of M14-359, respectively. LSP classifier algorithm was then applied to obtain the LSP determination for each sample.

Construction of the Classification Algorithm

The steps outlined below were utilized to generate the classification algorithm used to assign LSP status.

Samples Used for Construction of the Classification Algorithm and to Evaluate Association with Veliparib Benefit: Formalin-fixed, paraffin-embedded tumor samples from biopsies taken prior to entry onto veliparib clinical trial M10-898 and samples from subjects diagnosed with NSCLC from a tissue bank were used as training samples to construct the classification algorithm. The features of these training samples are described in Table 2.

TABLE 2
Reported Sample Characteristics Used for Centroid Development

| | M10-898 | Purchased NSCLC |
|---|---|---|
| Characteristic | Tissues | Tissue |
| Total No. of specimens | 73 | 47 |
| Tumor specimen histology^{a} | | |
| Squamous cell carcinoma | 40 | — |
| Non-squamous carcinoma | 33 | — |
| Adenocarcinoma | — | 39 |
| Large cell carcinoma | — | 1 |
| Adenosquamous carcinoma | — | 7 |
| Sex | | |
| Female | 24 | 16 |
| Male | 49 | 31 |
| Stage | | |
| Metastatic | 55 | — |
| Locally advanced | 18 | — |
| III | — | 16 |
| IV | — | 31 |
| Smoking | | |
| Nonsmoker | 11 | 16 |
| Smoker | 62 | 27 |
| Unknown | — | 4 |

For M10-898 histology was determined by pathologist at the original site where patient was enrolled.

Unsupervised Clustering: Expression levels of the LSP classifier genes were measured using the methods outlined in the section below.

First, Z-score normalization is applied to the log-transformed RPKM data of the 52 LSP genes (see Table 3 for the list of genes) before entering downstream analyses. As some RPKM values are 0, a fixed value such as 0.1 was added to the RPKM expression such that the data could be log 2 transformed. Z-scores from these log-transformed RPKM data for 52 genes were calculated for each cohort.

TABLE 3
Relative Contribution to LSP Subtype Assignment
of Each Gene According to the Centroid

| Gene.id | LSP− AD-Score | LSP+ Non-AD-Score |
|---|---|---|
| CDH5 | 0.3184 | −0.232 |
| DOK1 | 0.2947 | −0.2147 |
| PECAM1 | 0.2801 | −0.2041 |
| HYAL2 | 0.2776 | −0.2022 |
| CLEC3B | 0.2759 | −0.201 |
| NKX2-1 | 0.2708 | −0.1973 |
| DSC3 | −0.2525 | −0.184 |
| MGRN1 | 0.2468 | −0.1798 |
| ME3 | 0.2435 | −0.1774 |
| HOXD1 | 0.2056 | −0.1498 |
| MYO7A | 0.2045 | −0.149 |
| TRIM29 | −0.1916 | 0.1396 |
| CAPG | 0.1914 | −0.1394 |
| HPN | 0.1798 | −0.131 |
| CACNB1 | 0.1794 | −0.1307 |
| SFN | −0.1715 | 0.1249 |
| ICA1 | 0.1699 | −0.1238 |
| ALDH3B1 | 0.155 | −0.113 |
| MYBPH | 0.1509 | −0.1099 |
| PLEKHA6 | 0.1504 | −0.1096 |
| GJB5 | −0.1408 | 0.1026 |
| PIK3C2A | 0.1289 | −0.0939 |
| LIPE | 0.1238 | −0.0902 |
| LGALS3 | 0.1155 | −0.0842 |
| MAPRE3 | 0.107 | −0.0779 |
| HNF1B | 0.1047 | −0.0763 |
| NFIL3 | 0.1031 | −0.0751 |
| TFAP2A | −0.0997 | 0.0727 |
| LRP10 | 0.0963 | −0.0701 |
| ANTXR1 | 0.0935 | −0.0681 |
| BMP7 | −0.0924 | 0.0673 |
| TUBA4A | 0.0898 | −0.0655 |
| ICAM5 | 0.0806 | −0.0587 |
| CIB1 | 0.0777 | −0.0566 |
| PAICS | −0.0644 | 0.0469 |
| ACVR1 | 0.0606 | −0.0441 |
| INSM1 | 0.0552 | −0.0402 |
| CHGA | 0.047 | −0.0343 |
| ABCC5 | −0.0459 | 0.0334 |
| CDKN2C | 0.0437 | −0.0318 |
| TCP1 | 0.0279 | −0.0203 |
| SCD5 | 0.02 | −0.0146 |
| PAK1 | −0.0197 | 0.0143 |
| CYB5B | 0.0147 | −0.0107 |
| ITGA6 | −0.014 | 0.0102 |
| CBX1 | 0.0081 | −0.0059 |
| STMN1 | −0.0031 | 0.0023 |

After Z-score normalization, the K-means clustering algorithm with Manhattan distance was performed on 52 LSP genes of the training samples to produce 3 clusters. According to the definition of LSP, these three clusters should correspond to AD, NE, and SqCC.

Label Determination for Clusters: According to the definition of LSP, the three clusters produced should correspond to AD, NE, and SqCC. Concordance of each of the clusters with histologic-based subtyping (as extracted from local pathology diagnosis) was performed to determine which cluster corresponded to AD, NE, and SqCC. The concordance between molecular cluster and histology is shown in Table 4. SqCC histology was most frequently seen in cluster A, and non-squamous histology was most frequently seen in cluster C. Consequently, the SqCC label was assigned to cluster A; the AD label was applied to cluster C, and NE was assigned to cluster B.

TABLE 4
Label Determination for Three Clusters

| | Cluster A | | Cluster B | | Cluster C | |
|---|---|---|---|---|---|---|
| Histology | (SQ) | | (NE) | | (AD) | |
| Non-squamous cell carcinoma | 1 | (3%) | 9 | (27%) | 23 | (70%) |
| Squamous cell carcinoma | 28 | (70%) | 4 | (10%) | 8 | (20%) |
| Adenocarcinoma | 5 | (13%) | 17 | (44%) | 17 | (44%) |
| Adenosquamous carcinoma | 4 | (57%) | 1 | (14%) | 2 | (29%) |
| Large cell carcinoma | 0 | (0%) | 0 | (0%) | 1 | (100%) |

Centroid Development: The process of distilling the expression values of each of the LSP classifier genes into an LSP status utilizes the nearest shrunken centroid method. The list of genes and the shrunken centroids is shown in Table 3.

Determination of Assay Cutoff: The classification algorithm utilizes Z-scores from the log 2 transformed RPKM data of LSP genes to calculate a probability score based on likelihood of being LSP+ or LSP−. A sample is classified LSP+ if prob (nonAD)>=0.6; otherwise, the sample is LSP−. For any new samples, the resulting gene expression values are utilized and LSP status assigned using this classifier and cutoff.

The probability cutoff of 0.6 was derived from the complete Leave-One-Out Cross-Validation (LOOCV) procedure using the training samples (M10-898 and purchased tissue). In each step of the LOOCV, one sample was left out, and a classifier was trained based on the remaining samples following the same steps as discussed above (unsupervised clustering, subtype label determination, and centroid development using nearest shrunken centroid); the trained classifier was then applied to the leave-out sample to calculate its probability of subtype membership, e.g., prob (nonAD); these steps were repeated by leaving each sample one at a time, so that the probabilities of subtype memberships for all samples were obtained. The area under the receiver-operating characteristic curves (AUC) of prob (nonAD) is 0.94 (95% CI, 0.89, 0.98) in predicting the labels of (SQ+NE vs. AD). To derive the optimal cutoff, 3-subtype classifiers were trained, and one of the subtype calls for SQ, NE, and AD was assigned to each of the leave-out samples using the same LOOCV procedure. The optimal cutoff for prob (nonAD) was determined by maximizing the consistency between the LSP+vs. LSP− binary calling and the SQ+NE vs AD calling.

One feature of the LSP classification is that it identifies LSP+ cases that are a poor prognostic subgroup in histologically determined NSCLC AD. Therefore, in the TCGA cases assigned above, survival analysis was performed to verify that the constructed LSP classification algorithm can identify the poor performing subgroup. It concluded that the LSP classification algorithm can successfully identify patients who display a poor prognosis.

TABLE 5
List of Acronyms

| | |
|---|---|
| AD | adenocarcinoma |
| ALK | anaplastic lymphoma kinase |
| AUC | area under the receiver-operating characteristic curves |
| CDER | Center for Drug Evaluation and Research |
| CDRH | Center for Devices and Radiological Health |
| cDNA | complementary DNA |
| ds-cDNA | double-stranded-complementary DNA |
| EGFR | epidermal growth factor receptor |
| FDA | Food and Drug Administration |
| FFPE | formalin-fixed, paraffin-embedded |
| FISH | fluorescence in situ hybridization |
| H&E | hematoxylin and eosin |
| IHC | Immunohistochemistry |
| LOOCV | Leave-One-Out Cross-Validation |
| LSP | Lung Subtyping Panel |
| NCCN | National Comprehensive Cancer Network |
| NE | Neuroendocrine |
| NOS | not otherwise specified |
| NSCLC | non-small cell lung cancer |
| PARP | poly(adenosine diphosphate-ribose) polymerase |
| qRT-PCR | quantitative reverse transcription-polymerase chain reaction |
| RNAseq | RNA sequencing |
| RUO | Research Use Only |
| SCLC | small cell lung cancer |
| SQ | Squamous |
| TCGA | The Cancer Genome Atlas |

### Example 3

LSP Determination

RNA Isolation

Tumor samples for study were from biopsies taken prior to entry onto veliparib clinical trials M10-898 and M11-089. Tumor DNA/RNA was obtained by macrodissecting tumor area (>50% tumor content) from formalin-fixed, paraffin-embedded tumor slides. RNA and DNA was isolated using AllPrep kit (Qiagen).

RNAseq Library Preparation and Sequencing

Integrity of isolated RNA was performed using an agilent bioanalyzer and quantitated using picogreen. Library preparation was performed with 1-50 ng of total RNA. ds-cDNA was prepared using the SeqPlex RNA Amplification Kit (Sigma) per manufacturer's protocol. cDNA was blunt ended, had an A base added to the 3′ ends, and then had Illumina sequencing adapters ligated to the ends. Ligated fragments were then amplified for 12 cycles using primers incorporating unique index tags. Fragments were sequenced on an Illumina HiSeq-2500 or HiSeq-3000 using single reads extending 50 bases. 25-30M reads per library are targeted

RNA Sequencing (RNAseq) Data Analysis

RNAseq RPKM data of 52 LSP genes of Table 1 were obtained from sequenced samples of M10-898 clinical trial (https://clinicaltrials.gov/ct2/show/NCT01560104), purchased tissues and M11-089 (https://clinicaltrials.gov/ct2/show/NCT02106546). The RPKM data was log transformed and normalized in order to adjust for batch effects before entering the analyses. Unsupervised Clustering was performed on 52 LSP genes from M10-898 and purchased tissues to form 3 clusters of subtypes (AD (Adenocarcinoma), NE (Neuroendocrine), SQ (Squamous)), according to the consistency of known histology defined biologically distinct subtypes. The 52 LSP genes from M10-898 and purchased tissues together with the AD vs. nonAD labelling from unsupervised clustering is served as the training data. A shrunken centroid classifier is built based on the training data. The final classifier takes normalized RPKM data of 52 LSP genes as input, and outputs the probability of being nonAD subtype (prob (AD)=1-prob (nonAD)) for each RNAseq sample from M11-089. The subtype calls (nonAD vs. AD) is based on the probability strength. The subtype call will be labelled unknown If the absolute logarithm of odds (defined as Prob (nonAD)/Prob (AD)) is less than 0.15.

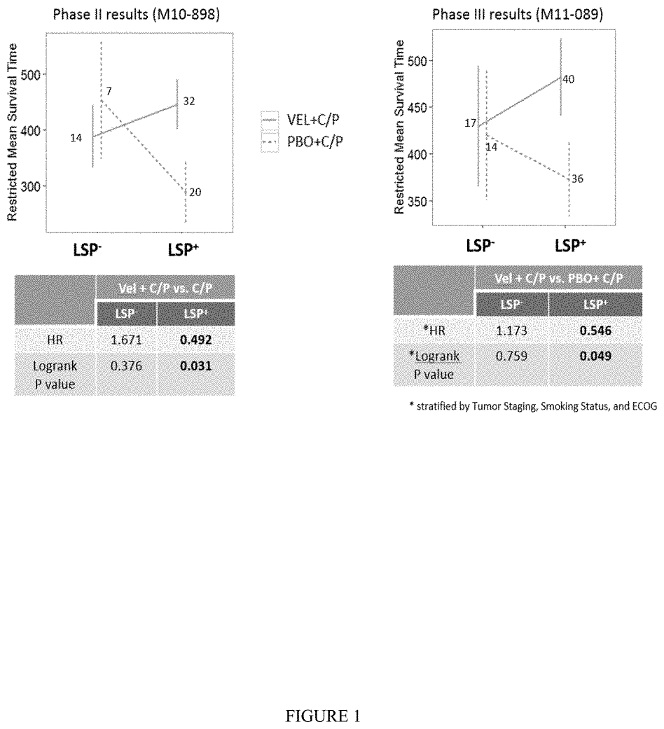

FIG. 1 represents the impact of treatment and patient outcome in M10-898 as a function of LSP status in M10-898 (left) and M11-089 (right). Patients were categorized as LSP− or LSP+ using RNA expression as depicted on the x-axis. Patients treated with carboplatin/paclitaxel (C/P) are listed in dotted lines, patients treated with C/P+veliparib (V) are listed in solid lines. The restricted mean survival time was calculated and is represented on the Y-axis. Summary hazard ratios (HR) for each comparison are also provided in tables underneath. Veliparib addition to C/P was associated with a statistically significant survival advantage only in the LSP+ population.

To locate the most critical LSP genes, we used the 47 genes ranked from most important to least important according to the PAMR algorithm that we used in the M10-898+ purchased tumor training model to perform a forward stepwise modeling to predict the LSP+vs. LSP− labels in training cohort, and the following is the cross-validated prediction error curve along with the number of additional genes. From the curve, after adding the first 12 genes, the CV classification error didn't decease much. The 12 critical LSP genes are (in order of prediction power): NKX2-1, DSC3, HPN, HNF1B, ALDH3B1, CDH5, DOK1, PECAM1, HYAL2, CLEC3B, MGRN1, and ME3.

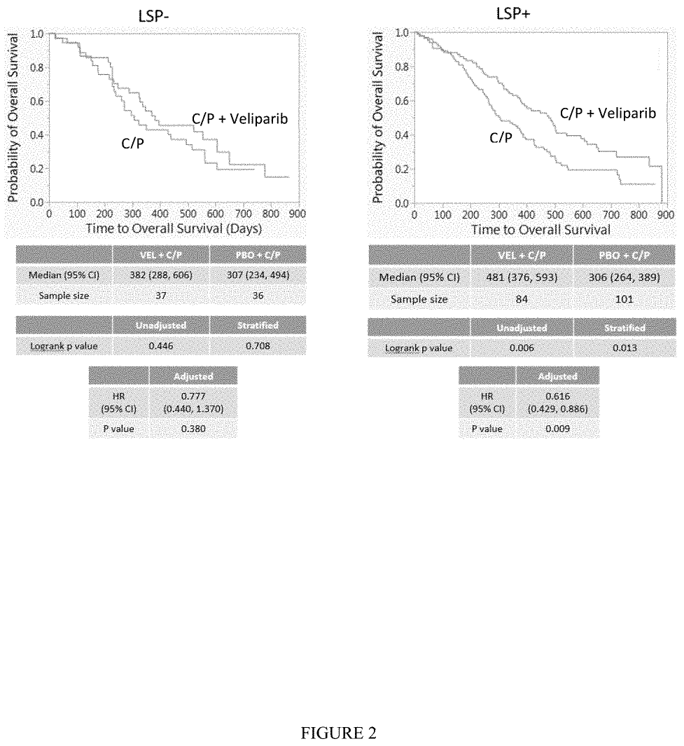

FIG. 2 shows median overall survival in LSP− (left) and LSP+ (right) patients from M11-089 treated with C/P alone or with C/P+veliparib. Summary statistics for each comparison are shown below FIG. 2. Veliparib addition to C/P was associated with a statistically significant survival advantage only in the LSP+ population.

Evaluations of LSP Markers

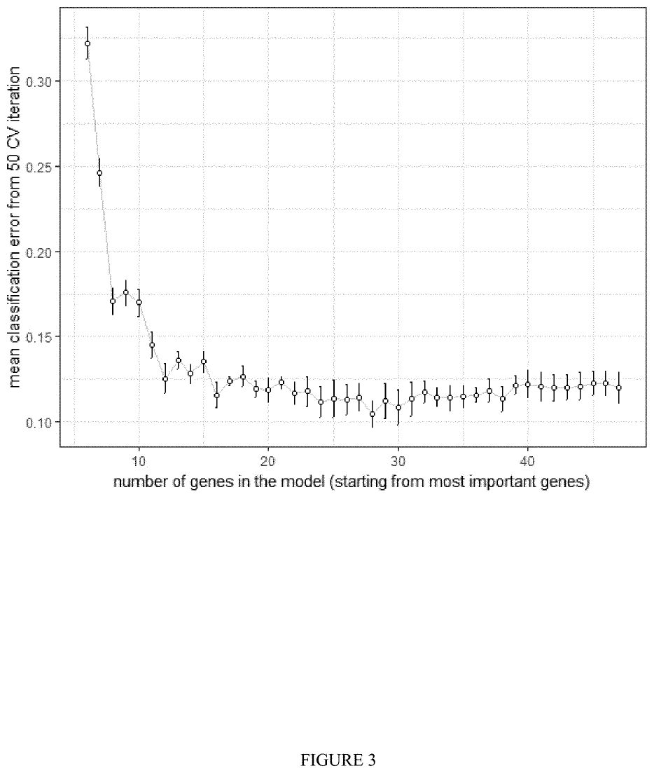

During the data analysis described above, it was found that not all LSP genes exhibited same contributions to LSP's predictive power. A serial of future evaluations was carried out to locate the most critical LSP markers by performing a forward stepwise modeling in M10-898 training models. As shown in FIG. 3, the classification errors decreased through a four-step process and eventually stabilized after 12 genes were used in prediction, and the 12 genes included NKX2-1, DSC3, HPN, HNF1B, ALDH3B1, CDH5, DOK1, PECAM1, HYAL2, CLEC3B, MGRN1 and ME3, which is in descending order with respect to importance, from most critical to least critical.

Stemness Score—mRNAsi vs LSP

We examined the M11-089 samples to relate the stemness index to survival with Veliparib. In general, an index ranging from 0 to 1 is created for each tumor sample. Tumor cells closer to 1 are more similar to stem cells and more aggressive than tumor cells closer to 0. We found that a stemness score of greater than or equal to 0.5 is associated with better survival with a PARP inhibitor, such as Veliparib.

We derived the stemness score from our samples based on stemness Index Derived using OCLR (mRNAsi) (T. M. Matta, et al., Cell, 2018, 173(2): 338-354), and methodology and code reference is based on the public available information (http://tcgabiolinks.fmrp.usp.br/PanCanStem/mRNAsi.html). The log gene expression for m11-089 is the batch effect corrected combined batch 1,2,3,4 data.

The results of our experiment are as follows in Table 6:

TABLE 6
Stemness Scores

| | | | | | | | Fisher's |
|---|---|---|---|---|---|---|---|
| | | Stemness Score | | | | Odds | Exact |
| | | NO | | YES² | | Ratio | Test |
| M11089 | LSP− | 146 | (77%) | 43 | (23%) | 3.28 | 9E−09 |
| | LSP+ | 131 | (51%) | 127 | (49%) | | |
| M14359 | LSP− | 74 | (76%) | 23 | (24%) | 5 | 3.21E−07 |
| | LSP+ | 35 | (38%) | 55 | (62%) | | |
| LUSC | LSP− | 127 | (64%) | 70 | (36%) | 4.9 | <2E−16 |
| | LSP+ | 81 | (27%) | 223 | (73%) | | |
| LUAD | LSP− | 197 | (81%) | 46 | (19%) | 7.2 | <2E−16 |
| | LSP+ | 101 | (37%) | 171 | (63%) | | |

²Stemness score >=0.5

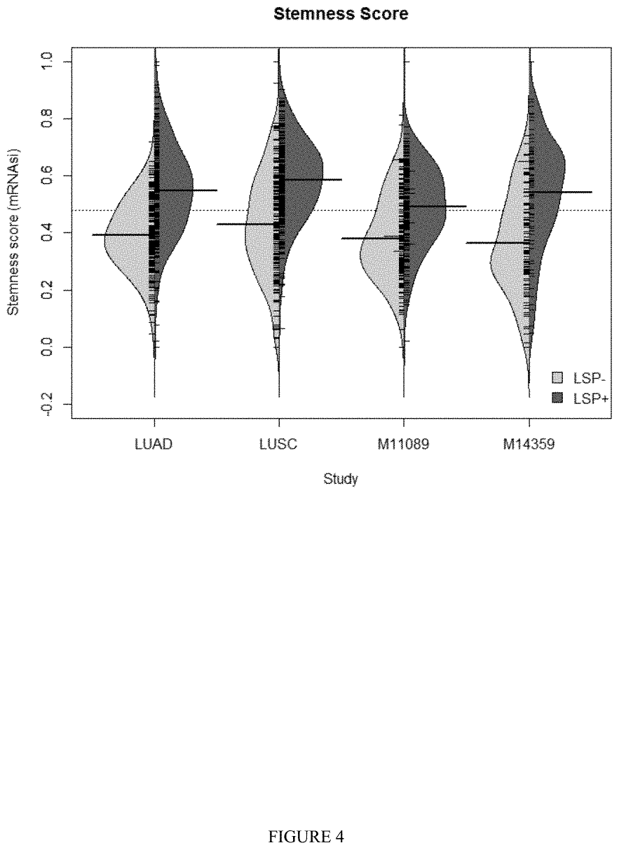

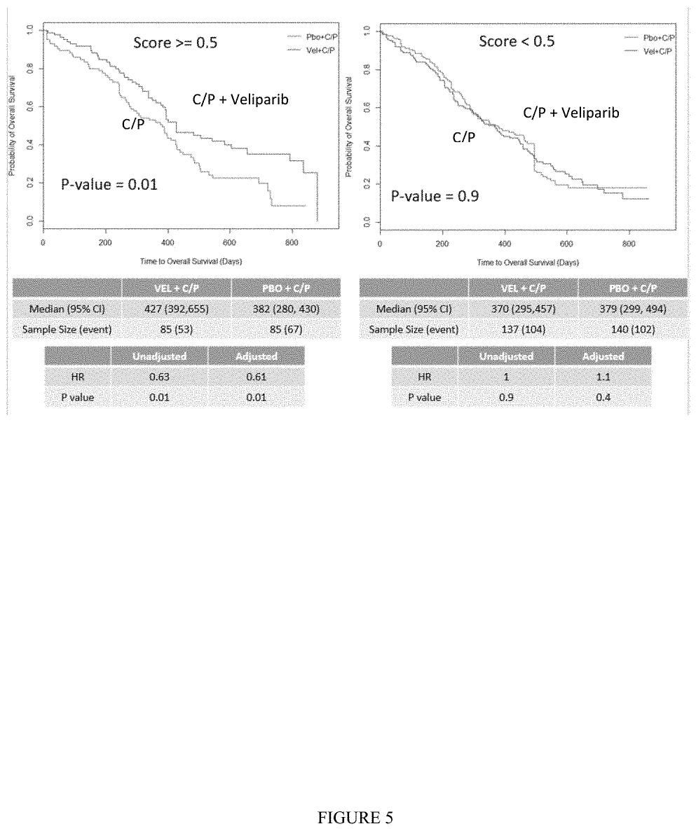

LSP+ population was characterized as having a high stemness score (>=0.5). Higher stemness score (>=0.5) is associated with better survival with veliparib treatment as demonstrated in FIGS. 4 and 5.

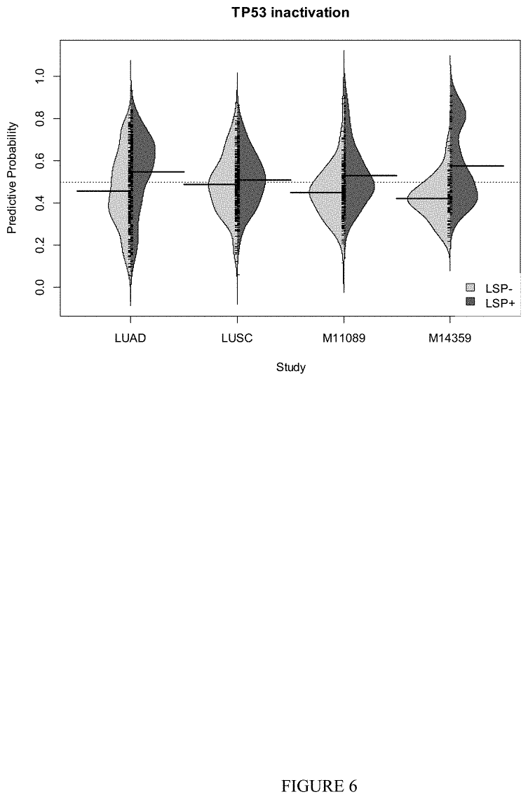

LSP+ tumors were characterized by their increased P53 inactivation. Similar to our stemness analysis, in-silico expression based TP53 inactivation was performed as described in Knijnenburg et al, 2018 to assess the TP53 deficiency (TP53 inactivation) in the tumor types. As shown in FIG. 6 and Table 7 below, a LSP+ score is correlated with TP53 deficiency.

TABLE 7
TP53 Deficiency

| | | | | | | | Fisher's |
|---|---|---|---|---|---|---|---|
| | | TP53 deficiency¹ | | | | Odds | Exact |
| | | NO | | YES² | | Ratio | Test |
| M11-089 | LSP− | 130 | (70.3%) | 55 | (29.7%) | 2.40 | 1.66E−05 |
| | LSP+ | 129 | (49.6%) | 131 | (50.4%) | | |
| M14-359 | LSP− | 75 | (82.4%) | 16 | (17.6%) | 6.22 | 5.52E−08 |
| | LSP+ | 35 | (42.7%) | 47 | (57.3%) | | |
| LUSC | LSP− | 111 | (56.3%) | 86 | (43.7%) | 1.43 | 0.05489 |
| | LSP+ | 144 | (47.4%) | 160 | (52.6%) | | |
| LUAD | LSP− | 140 | (57.6%) | 103 | (42.4%) | 2.74 | 3.16E−08 |
| | LSP+ | 90 | (33.1%) | 182 | (66.9%) | | |

¹Knijnenburg et al, 2018
²predictive probability >0.5 from in-silico expression based TP53 inactivation predictor

Proliferation Score

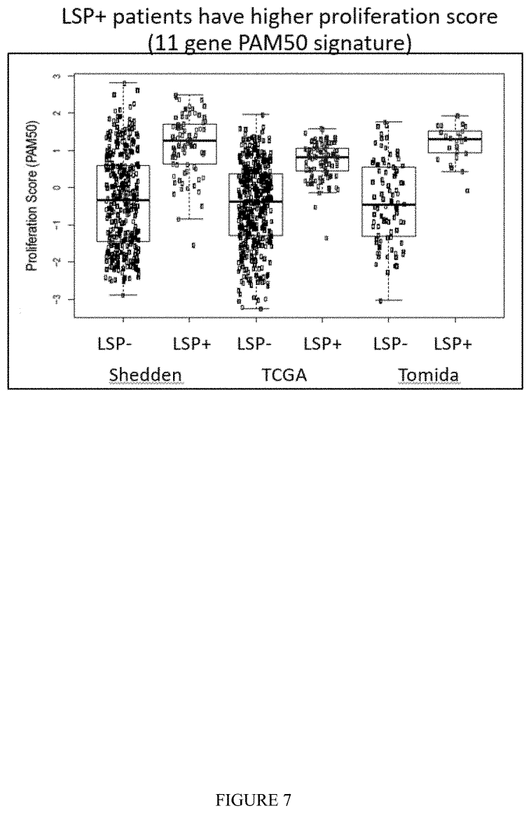

LSP+ patients can also be characterized as having a higher proliferation score (greated than 0). As demonstrated in FIG. 7, the proliferation score was determined using 11 gene PAM50 signature, and LSP+ tumors have higher proliferation scores (>0).

LSP+ Subtype Across Each Histological/Molecular Subtype

Comparison of the separation of tumors into adenocarcinoma, squamous, and small cell carcinoma and the LSP+ and LSP− populations demonstrate that LSP subtype is found in each of the histological groupings. As such, the LSP+ subpopulation is a unique population that cannot be determined by histology alone.

The present invention is not intended to be limited to the foregoing examples but encompasses all such modifications and variations as come within the scope of the appended claims.

We claim:

1. A method of treating non-small cell lung cancer in a patient comprising
administering to the patient an effective amount of a PARP inhibitor,
wherein the patient is Lung Subtyping Panel (LSP) positive.

2. The method of claim 1, wherein the LSP positive patient has a proliferation index higher than 0.

3. The method of any one of claims 1-2, wherein the LSP positive patient has a stemness score of at least 0.5.

4. The method of any one of claims 1-3, wherein the LSP positive patient has TP53 deficiency of at least 50%.

5. The method of claim 1, wherein the LSP positive patient is positive for one or more LSP markers.

6. The method of claim 5, wherein the LSP markers comprise at least one of NKX2-1, DSC3 and HPN.

7. The method of claim 6, wherein the LSP markers additionally comprise HNF1B or ALDH3B1, or both.

8. The method of any one of claims 6-7, wherein the LSP markers additionally comprise at least one marker selected from a group consisting of CDH5, DOK1, PECAM1, HYAL2 and CLEC3B.

9. The method of any one of claims 6-8, wherein the LSP markers additionally comprise MGRN1 or ME3, or both.

10. The method of any one of claims 6-9, wherein the LSP markers comprise at least one markers selected from a group consisting of NKX2-1, DSC3, HPN, HNF1B, ALDH3B1, CDH5, DOK1, PECAM1, HYAL2, CLEC3B, MGRN1 and ME3.

11. The method of any one of claims 6-10, wherein the LSP markers optionally comprises one or more additional genes selected from a group consisting of ABCC5, ACVR1, ANTXR1, BMP7, CACNB1, CAPG, CBX1, CDKN2C, CFL1, CHGA, CIB1, CYB5B, EEF1A1, FEN1, FOXH1, GJB5, HOXD1, ICA1, ICAM5, INSM1, ITGA6, LGALS3, LIPE, LRP10, MAPRE3, MYBPH, MYO7A, NFIL3, PAICS, PAK1, PIK3C2A, PLEKHA6, PSMD14, RPL10, RPL28, RPL37A, SCD5, SFN, SIAH2, SNAP91, STMN1, TCP1, TFAP2A, TRIM29 and TUBA4A.

12. The method of claim 1, wherein the PARP inhibitor is a PAPR-1 inhibitor, a PARP-2 inhibitor, or a PARP-1/2 inhibitor.

13. The method of claim 12, wherein the PARP inhibitor is selected from a group consisting of veliparib, niraparib, olaparib, rucaparib and talazoparib.

14. The method of claim 13, wherein the PARP inhibitor is veliparib.

15. The method of claim 1, wherein the PARP inhibitor is administered in combination with one or more chemotherapy agents.

16. The method of claim 15, wherein the chemotherapy agent is carboplatin.

17. The method of claim 15, wherein the chemotherapy agent is paclitaxel.

18. The method of claim 15, wherein the PARP inhibitor is administered in combination with carboplatin and paclitaxel.

19. The method of claim 18, wherein the PARP inhibitor is veliparib.

20. The method of claim 19, wherein veliparib is administered 120 mg, twice a day.

21. The method of claim 18, wherein carboplatin is administered AUC 6 mg/mL·min.

22. The method of claim 18, wherein paclitaxel is administered 200 mg/m².

23. The method of claim 1, wherein the LSP patient has squamous NSCLC.

24. The method of claim 1, wherein the LSP patient has non-squamous NSCLC.

25. The method of claim 1, wherein the LSP patient has advanced NSCLC.

26. The method of claim 1, wherein the LSP patient has metastatic NSCLC.

27. The method of claim 1, wherein the LSP patient is not tested positive for EGFR, ALK or ROS mutation.

28. The method of claim 1, wherein the LSP patient has tumor proportion score (TPS) of lower than 50% for PD-L1.

29. The method of any one of the proceeding claims, wherein the method increases overall survival of the LSP positive patient by at least 5 months.

30. The method of any one of the proceeding claims, wherein the method increases progression-free survival by at least 2.5 months.
